(19) United States
(12) Patent Application Publication
(10) Pub. No.: US 2020/0202982 A1
(43) Pub. Date: Jun. 25, 2020

(54) METHODS AND SYSTEMS FOR ASSESSING THE PRESENCE OF ALLELIC DROPOUT USING MACHINE LEARNING ALGORITHMS

(71) Applicants: Michael Marciano, Manlius, NY (US); Jonathan D. Adelman, Mexico, NY (US)

(72) Inventors: Michael Marciano, Manlius, NY (US); Jonathan D. Adelman, Mexico, NY (US)

(73) Assignee: SYRACUSE UNIVERSITY, SYRACUSE, NY (US)

(21) Appl. No.: 16/612,647

(22) PCT Filed: May 17, 2018
(86) PCT No.: PCT/US18/33154

§ 371 Date: Nov. 11, 2019

Related U.S. Application Data
(60) Provisional application No. 62/507,413, filed on May 17, 2017.

(51) Int. Cl. G16B 40/00; G16B 30/00; G16B 50/30; G06N 20/10; G06F 17/18

(52) U.S. Cl. CPC G16B 40/00; G16B 30/00; G06F 17/18; G06N 20/10; G16B 50/30

(57) ABSTRACT

A system configured to characterize the probability of any allele dropout in the sequence of DNA extracted from a sample. The system includes a sample preparation module that can generate sequence data about any DNA within the sample, a processor that is programmed to receive the sequence data and determine the probability of allelic dropout in the sequence data, and an output device that provides the determination of allele dropout to a user of the system.

### CROSS-REFERENCE TO RELATED APPLICATIONS

This application claims priority to U.S. Provisional Application No. 62/507,413, filed on May 17, 2017.

### STATEMENT REGARDING FEDERALLY SPONSORED RESEARCH OR SUPPORT

This invention was made with government support under Grant No. 2014-dn-bx-k029, awarded by the National Institute of Justice. The government has certain rights in the invention.

### BACKGROUND OF THE INVENTION

### 1. Field of the Invention

The present disclosure is directed generally to methods and systems for identifying nucleic acid in a sample and, more particularly, to methods and systems for characterizing the presence of allelic dropout in a DNA sample.

### 2. Description of the Related Art

Genetic identification remains a tenet of many private and public sectors, from food science to enology, oncology and forensic science and national security matters. The quality of the analyses and interpretation of this genetic information can directly impact the confidence in the resulting conclusions.

In the context of genetic identity testing, a DNA sample can be defined as a sample containing the DNA of one or more individuals. The variety of subtypes of DNA samples can lead to interpretational challenges, particularly in the context of criminal investigations or sensitive site exploitation. One such challenge is the interpretation of low quantities of DNA, termed low template DNA analysis. When an individual's DNA is present at exceedingly low levels within the sample, it is possible that genetic information is absent due to stochastic effects e.g. sampling bias. This phenomenon, where the expected allelic information is not represented in a DNA sample, is known as allele dropout. Allelic dropout is a well-known phenomenon in genetic identification. Dropout is most commonly observed in low template DNA samples, DNA mixtures where one or more of the components have low levels of DNA template, and in samples with inhibition. The presence of allelic dropout can be further influenced by technology used to analyze the raw data and algorithms used to process the electronic data.

The assessment of allelic dropout is most critical when interpreting a mixed DNA sample. A mixed DNA sample can be defined as a mixture of two or more biological samples. The analysis and interpretation of DNA mixture samples have long been a challenge area in genetic identification and mastery of their interpretation could greatly impact the course of criminal investigations and/or quality of intelligence. The inability to account for allelic dropout may lead to erroneous conclusions, and many times lead to inconclusive results.

Several metrics are critical to predicting allelic dropout, including but not limited to the quantitative measure of an allele (peak heights (rfu) or allele counts), the estimated DNA template used for preprocessing (PCR), the estimated number of contributors and the estimate ratio of DNA contributions by each donor (when the sample is a DNA mixture). The method also utilizes additional metrics such as the mean and standard deviation of the allelic representation across a locus (peak height or count), the average peak area divided by the average peak height, the height or count of the highest and lowest represented allele at a locus and related ratio.

Significant efforts have been placed on modeling allelic dropout and have yielded useful tools in addressing the interpretation of DNA samples. However, these methods have been limited by the limited number of components used to predict allelic dropout. Increasing the accuracy of detection would serve to improve the final interpretation.

Accordingly, there is a need in the art for methods and systems that perform complicated DNA sample interpretation, particularly with regard to improving the detection and assessment of allelic dropout.

### BRIEF SUMMARY OF THE INVENTION

The present invention is directed to methods and systems for identifying instances of allelic dropout during the course of DNA analyses. The method and systems described herein probabilistically infer the presence of allele dropout using a machine learning approach. Classification problems involving machine learning contain a learning phase, in which training data are used to inform the learning algorithm, and a modeling phase, in which the informed algorithm creates a predictive model. Such a model requires a vector of features, which are measurable properties or characteristics of an observed phenomenon. The conclusions generated about allelic dropout by the present invention are based on the use of both categorical (qualitative) data such as allele labels, dye channels and continuous and discrete (quantitative) data such as stutter rates, peak heights, heterozygote balance, and mixture ratios that describe the DNA sample. The present invention is capable of returning results in seconds once the predictive module is determined, is computationally inexpensive, and can be performed using a conventional hardware, such as standard desktop or laptop computer with off-the-shelf processors.

In an embodiment, the invention may be a system configured to characterize allele dropout in a sample. The system has a processor programmed to receive sequence data representing DNA in the sample and to predict the occurrence of any allelic dropout at a given locus by applying a machine-learning algorithm to assess the categorical and quantitative aspects of the sequence data. The system also has an output device configured to receive the predicted occurrence of allele dropout from the processor and provide the predicted occurrence to a user. The machine-learning algorithm may be a support vector machine algorithm. The output device may be a monitor. The sample preparer may be configured to generate the sequence data about DNA within the sample. The sample preparer may be configured to amplify DNA within the sample. The sample preparer may be configured to amplify at least one DNA marker within the sample.

In another embodiment, the invention may be a method of characterizing any occurrence of allele dropout in a sample. In a first step, the method includes using a sample preparer to generate sequence data for any DNA within the sample. In a second step, the method includes receiving the sequence data with a processor programmed to receive the sequence data. In a third step, the method includes using the processor to predict the occurrence of any allelic dropout at a given locus in the sequence data by applying a machine-learning algorithm to assess the categorical and quantitative aspects of the sequence data. In a fourth step, the method includes using an output device to receive the predicted occurrence of allele dropout from the processor and provide information about the received predicted occurrence of allele dropout to a user. The machine-learning algorithm may be a support vector machine algorithm. The output device may be a monitor. The first step may include the amplification of DNA within the sample. The first step may also include amplification of one or more DNA markers within the sample.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic representation of a system for DNA analysis, in accordance with an embodiment;

FIG. 2 is a schematic representation of a system for DNA analysis, in accordance with an embodiment;

FIG. 3 is an electropherogram used to demonstrate stutter calculations;

FIG. 4 is a graph of the percentage of accurately detected alleles resulting from the thresholding and noise reducing systems using internally developed stutter models and stock stutter models obtained from the developmental validation;

FIG. 5 is a graph of the percentage of additional non-allelic peaks detected by the thresholding and noise reducing systems using internally developed stutter models ( ) and stock stutter models;

FIG. 6 is a graph of the comparison of the number of incorrectly called alleles detected when trimming is applied to the thresholding method, trimming/noise reduction used, no trimming/noise reduction used;

FIG. 7 is a graph of the learning curve for the support vector machine used for initial classification of alleles, where shaded areas represent +/−one standard deviation;

FIG. 8 is a graph of the ROC curve for the support vector machine used for initial classification of alleles;

FIG. 9 is a graph of the distribution of the proportion of the detected alleles across threshold/NR methods and the number of contributors; and

FIG. 10 is a graph of the distribution of the proportion of the additional alleles across threshold/NR methods and the number of contributors.

### DETAILED DESCRIPTION OF THE INVENTION

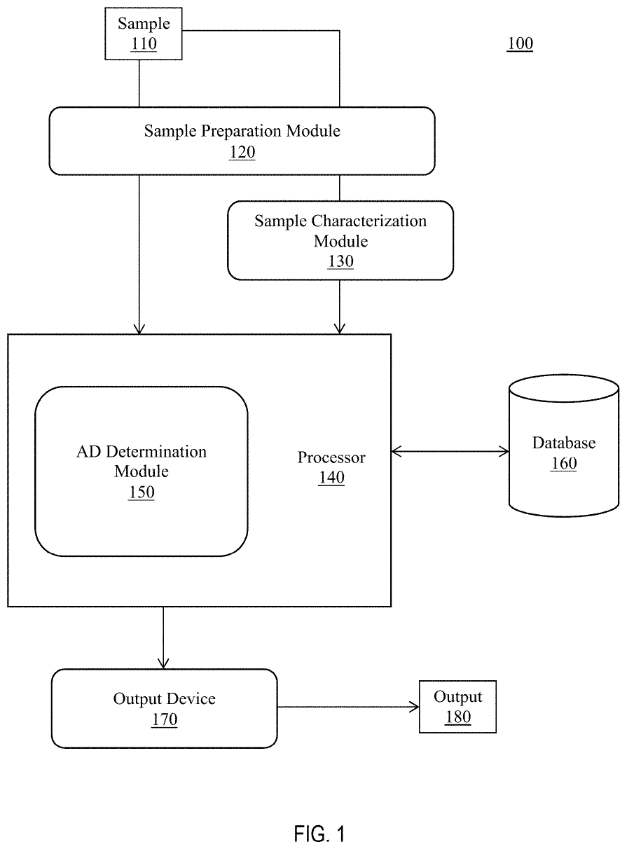

Referring to the figures, wherein like numeral refer to like parts throughout, there is seen in FIG. 1 a system that can perform complex DNA sample interpretation in both a time-effective and cost-effective manner. More specifically, the invention is directed to methods and systems for assessing the presence of allele dropout in a sample using machine learning approaches. The conclusions generated are based on the use of both qualitative data such as the number of alleles present at a locus and across a sample and discrete data such as the quantitative measure of an allele (peak heights (rfu) or allele counts), the estimated DNA template used for preprocessing (PCR), the estimated number of contributors and the estimate ratio of DNA contributions by each donor (when the sample is a DNA mixture). The method also utilizes additional metrics such as the mean and standard deviation of the allelic representation across a locus (peak height or count), the average peak area divided by the average peak height, the height or count of the highest and lowest represented allele at a locus and related ratio. The method is computationally inexpensive, and results are obtained within seconds using a standard desktop or laptop computer with a standard processor

According to an embodiment, the method employs a machine learning algorithm for one or more steps. Machine learning refers to the development of systems that can learn from data. For example, a machine learning algorithm can, after exposure to an initial set of data, be used to generalize; that is, it can evaluate new, previously unseen examples and relate them to the initial training data. Machine learning is a widely-used approach with an incredibly diverse range of applications, with examples such as object recognition, natural language processing, and DNA sequence classification. It is suited for classification problems involving implicit patterns, and is most effective when used in conjunction with large amounts of data. Machine learning might be suitable for the prediction of allelic dropouts, as there are large repositories of human DNA sample data in electronic format. Patterns in this data are often non-obvious and beyond the effective reach of manual analysis, but can be statistically evaluated using one or more machine learning algorithms as described or otherwise envisioned herein.

Referring to FIG. 1, in one embodiment, is a system 100 for characterizing the level of allelic dropout within a sample 110, where sample potentially 110 contains DNA from one or more sources. Sample 110 can previously be known to include a DNA sample or a mixture DNA sample of DNA from two or more sources, or can be an uncharacterized sample. Sample 110 can be obtained directly in the field and then analyzed, or can be obtained at a distant location and/or time prior to analysis. Any sample that could possibly contain DNA therefore could be utilized in the analysis.

According to an embodiment, system 100 can comprise a sample preparer 120. Sample preparer 120 can be a combination of DNA sequencing devices and systems that prepares the obtained sample for DNA analysis. For example, sample preparer 120 may comprise systems that can perform DNA isolation, extraction, separation, and/or purification. According to an embodiment, sample preparer 120 may include modifications of the sample to prepare that sample for analysis according to the invention.

According to an embodiment, system 100 can optionally comprise a sample characterizer 130. For example, DNA present in the sample can be characterized by, for example, capillary electrophoresis based fragment analysis, sequencing using PCR analysis with species-specific and/or species-agnostic primers, SNP analysis, one or more loci from human Y-DNA, X-DNA, and/or atDNA, or any other of a wide variety of DNA characterization methods. According to advanced methods, other characteristics of the DNA may be analyzed, such as methylation patterns or other epigenetic modifications, among other characteristics. According to an embodiment, the DNA characterization results in one or more data files containing DNA sequence and/or loci information that can be utilized for identification of one or more sources of the DNA in the sample, either by species or individually within a species (such as a particular human being, etc.). Commonly used features such as total DNA amplified, peak height, sequence count, presence of single nucleotide polymorphisms, phred score, and sequence length variants should be included as part of the characterization for consideration by the machine leaning algorithm of prediction module 150, as described herein. Sample characterizer 130 may include a feature extraction module configured to extract high-information features from the DNA sample, including features not used by those skilled in the art for the characterization of nucleic acids in a sample. For example, various derived features unique to the present invention may also be considered, such as: the number of contributors estimated (maximum and minimum) from prior machine learning algorithms, peak height ratios (the relative contributions of each contributor in a mixed DNA profile estimated using a unique clustering method), a mixture ratio metric representing similarity between the calculated mixture ratio for each genotype combination and the sample-wide mixture ratio obtained via clustering (inter and intra-locus peak height/intensity ratios), a signal balance metric representing how in balance contributors in a genotype combination are to one another (inter and intra locus peak height/count balance), results from a unique signal detection tool that is implemented per DNA locus i.e. marker (within a sample) (locus specific count/peak amplitude threshold-), number of signals trimmed by the signal detection tool (artifacts such pull-up, spikes, sequence errors), peak height or sequence count of a bi-allelic gender determining marker divided by the total peak height or sequence count of a multi-allelic, gender specific marker divided by the number of contributors as determined using the maximum allele count method, probability of allelic dropin (allelic dropin) and weighted deconvoluted genotypes.

According to an embodiment, system 100 comprises a processor 140. Processor 140 can comprise, for example, a general purpose processor, an application specific processor, or any other processor suitable for carrying out the sequence data and machine learning analysis processing steps as described or otherwise envisioned herein. According to an embodiment, processor 140 may be a combination of two or more processors. Processor 140 may be local or remote from one or more of the other components of system 140. For example, processor 140 might be located within a lab, within a facility comprise multiple labs, or at a central location that services multiple facilities. According to another embodiment, processor 140 is offered via a software as a service. One of ordinary skill will appreciate that non-transitory storage medium may be implemented as multiple different storage mediums, which may all be local, may be remote (e.g., in the cloud), or some combination of the two.

According to an embodiment, processor 140 comprises or is in communication with a non-transitory storage medium, such as a database 160. Database 160 may be any storage medium suitable for storing program code for executed by processor 140 to carry out any one of the steps described or otherwise envisioned herein. Database 160 may be comprised of primary memory, secondary memory, and/or a combination thereof. As described in greater detail herein, database 160 may also comprise stored data to facilitate the analysis, characterization, and/or identification of the DNA in the sample 110.

According to an embodiment, processor 140 is programmed to include an allelic dropout (AD) prediction module 150. Allelic dropout algorithm or module 150 may be configured to comprise, perform, or otherwise execute any of the functionality described or otherwise envisioned herein. According to an embodiment, AD determination algorithm or module 150 receives data about the DNA within the sample 110, among other possible data, and utilizes that data to predict or determine the occurrence of allelic dropout of DNA within that sample, among other outcomes. According to an embodiment as described in detail herein, AD determination algorithm or module 150 comprises a trained or trainable machine-learning algorithm configured or configurable to predict the occurrence of allelic dropout within sample 110. For example, the machine learning algorithm is trained to develop an allelic dropout model using known occurrences of allelic dropout to identify which extracted features to consider and how to account for the features in the allelic dropout model.

Processor 140 is additionally programmed to implement the allelic dropout model to probabilistically determine if allele dropout is present at a DNA locus of interest in an unknown sample. The probability reflects the chance of information being expected but not present at a particular locus. Knowing this information is critical to the ability to accurate interpret the DNA sample of interest. In this embodiment, database 160 will need to include a comprehensive data set of known samples with correctly labeled nucleic acids, to be used in the training, calibrating, testing, and validation of a machine learning algorithm 150. Machine learning algorithm for prediction module 150 is configured to accept input of data from a data repository module that has been compressed through the use of a feature extraction module, and further configured to utilize one or more machine learning algorithms to best learn an optimized, predictive model capable of characterizing the probability of allelic dropout at any given locus in a sample, whereby the input into a machine learning algorithm is the feature vector created from the feature extraction module. For an unknown sample, a prediction module 150 is programmed to use the optimized, predictive model initially learned by prediction module 150 during configuration (or provided with a validated algorithm as a configuration file), to receive as input any new sample previously unexposed to the system, and then to produce as output the probability of allelic dropout occurring at a given locus for the sample. The machine learning algorithm that may be used as part of prediction module 150 include artificial neural networks such as a multi-layer perceptron, support vector machines, decision trees such as C4.5, ensemble methods such as stacking, boosting and random forests, deep learning methods such as a convolutional neural network, and clustering methods such as k-means. Prediction module 150 may thus be used to train a machine learning algorithm to identify allelic dropout using known sample data in database 160, to apply a trained machine learning algorithm to identify allelic dropout in unknown sample data in database 160, or both.

According to an embodiment, system 100 comprises an output device 170, which may be any device configured to or capable of generating and/or delivering output 180 to a user or another device. For example, output device 170 may be a monitor, printer, or any other output device. The output device 170 may be in wired and/or wireless communication with processor 140 and any other component of system 100. According to yet another embodiment, the output device 170 is a remote device connected to the system via a network. For example, output device 170 may be a smartphone, tablet, or any other portable or remote computing device. Processor 140 is optionally further configured to generate output deliverable to output device 170, and/or to drive output device 170 to generate and/or provide output 180.

As described herein, output 180 may comprise information about the level of allele dropout found in the sample, and/or any other received and/or derived information about the sample.

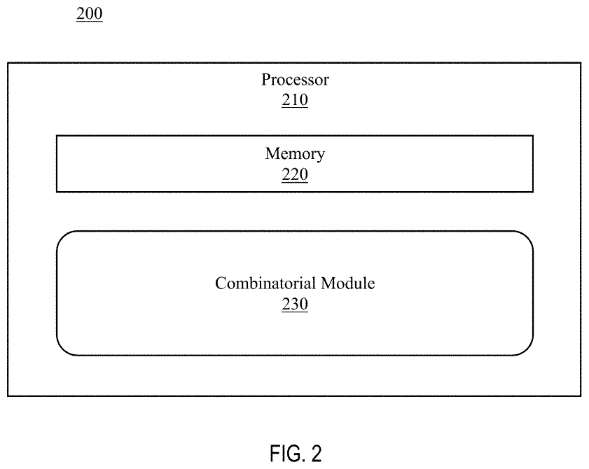

Referring to FIG. 2, in one embodiment, is a schematic representation of a system 200 for characterizing the level of allele dropout within a sample. The sample can previously be known to include a DNA sample or a mixture of DNA from two or more sources, or can be an uncharacterized sample. The sample can be obtained directly in the field and then analyzed, or can be obtained at a distant location and/or time prior to analysis. Any sample that could possibly contain DNA therefore could be utilized in the analysis.

According to an embodiment, system 200 comprises a processor 210. Processor 210 can comprise, for example, a general purpose processor, an application specific processor, or any other processor suitable for carrying out the processing steps as described or otherwise envisioned herein. According to an embodiment, processor 210 may be a combination of two or more processors. Processor 210 may be local or remote from one or more of the other components of system 210. For example, processor 210 might be located within a lab, within a facility comprise multiple labs, or at a central location that services multiple facilities. According to another embodiment, processor 210 is offered via a software as a service. One of ordinary skill will appreciate that non-transitory storage medium may be implemented as multiple different storage mediums, which may all be local, may be remote (e.g., in the cloud), or some combination of the two.

According to an embodiment, processor 210 includes or is coupled to a non-transitory storage medium, such as a database 220. Database 220 may be any storage medium suitable for storing program code for executed by processor 210 to carry out any one of the steps described or otherwise envisioned herein. Database 220 may be comprised of primary memory, secondary memory, and/or a combination thereof. As described in greater detail herein, database 220 may also comprise stored data to facilitate the analysis, characterization, and/or identification of the DNA in the sample.

According to an embodiment, processor 210 comprises an allelic dropout (AD) module 230. AD determination module 230 may be configured to comprise, perform, or otherwise execute any of the functionality described or otherwise envisioned herein. According to an embodiment, AD determination algorithm or module 230 receives data about the DNA within a sample, among other possible data, and utilizes that data to predict or determine the occurrence of allele dropout within that sample, among other outcomes. According to an embodiment as described in greater detail herein, AD determination algorithm or module 230 comprises a trained or trainable machine-learning algorithm configured or configurable to determine the occurrence of allele dropout within a sample.

The methodology is computationally inexpensive and results can be obtained in 5 seconds or less using, for example, a standard desktop or laptop computer with 6-8 GB RAM and an Intel i5 1.9 gHz processor programmed to implement the modules of the present invention, although many other computational parameters are possible, including both significantly smaller and greater RAM, and/or significantly slower and faster processing speeds. According to an embodiment, the method achieves this through the use of machine learning and, in contrast to approaches that use traditional regression methods, leverages an initial training and testing data set to build the model. According to an embodiment, this imparts both speed and reproducibility onto the end user, with all of the computational heavy lifting done during data acquisition and model creation.

### Example

The invention was validated using 1301 single source and mixture samples from 1 to 4 contributors, which were amplified (28 cycles) using the PowerPlex Fusion Human DNA amplification kit (Promega Corporation). These samples were previously run on the Applied Biosystems 3100, 3130 and 3500 series of Genetic Analyzers (ThermoFisher Scientific Inc.) across 6 laboratories. The 3100 and 3130 sample injection times were at 5 s with injection voltages of 3, 6, 9 and 12 kV. Samples analyzed on the 3500 Genetic Analyzer were injected at 10, 15, 18 and 24 s with voltages of 1.2 and 12 kV. Electropherograms were analyzed using GeneMarkerHID v2.8.2 (SoftGenetics LLC) with a threshold of 10 RFU without stutter filters. Pull-up peaks were removed manually prior to data export; the identification of pull-up artifacts will be addressed in future versions. The data were exported from GeneMarkerHID v2.8.2 and processed using automated and intelligent locus-sample-specific threshold and noise reduction (iLSST-NR). Samples were processed using standard Windows 10 laptops (minimum specification: Intel i7-7500 2.7 Ghz 8 MB RAM). The iLSST-NR pipeline analyzed samples in an average of 5.2±0.78 seconds.

The iLSST-NR method uses four distinct modules to detect alleles and remove artifacts. Module 1 imposes a dynamic analytical threshold to detect alleles and remove low-level noise. This dynamic threshold is calculated for each locus within a sample and is thus termed a locus-sample-specific threshold (LSST). Module 2 applies forward and reverse stutter filters to the remaining data (after the application of the LSST). Module 3 consists of trimming algorithms that remove noise that may have been incorrectly classified following the application of the LSST. In the context of this invention, trimming refers to removal of a peak or noise from a locus. The fourth module consists of machine learning-derived models used to detect and assess probabilistically the presence of additional incorrectly detected noise in a locus. These resulting probabilities are used to further remove incorrectly detected noise or artifacts prior to a final feature vector being input into a final module consisting of a machine learning-derived model used to probabilistically predict the presence of dropout at each locus in the data.

Module 1: Dynamic Threshold—Locus-Sample Specific Threshold (LSST)

The iLSST-NR system uses a dynamic analytical threshold, calculated based on the mean and standard deviations of the noise in regions flanking a locus within an individual sample. The goal of module 1, the dynamic threshold, is to avoid false negatives (i.e. removing true peaks) regardless of how many false positives (i.e. artifacts labeled as peaks) occur. Specific algorithms have been designed to identify and remove those false positives. Import requirements include CE-fragment data that have had the spectral calibration/matrix applied. The trace data obtained from the export of peak calling programs such as GeneMarker HID, GeneMapper HID (Applied Biosystems) or Osiris are used to calculate the threshold as described hereafter. Flanking regions are identified using a locus threshold dictionary, the values of which can be changed by the user. The mean and standard deviation of the y-coordinate data (height, in RFU) are calculated using the inter-locus ranges specified in the locus threshold dictionary, and an analytical threshold is set at four standard deviations above the mean. The dynamic threshold could be artificially elevated due to the presence of artifacts, such as pull-up, in the inter-locus regions. Pull-up and electrical spikes are detected and removed using a peak detection algorithm, with additional artificially raised baseline subject to a maximum RFU cap.

Module 2: Stutter

Forward and reverse stutter thresholds were applied using internally developed, empirically-derived stutter models using either a Gompertz function (Equation 1) or an exponential rise to maximum (Equation 2).

f(x)=ae−^{-be}^{^{cx}} (1)

f(x)=a−be^{cx} (2)

Additional filtering and trimming are performed based on the manufacturer's recommendations and developmental validation, for example, “non-traditional” stutter artifacts such as the n−1 peak at D2S441, n−2/n+2 peaks at D19S443 and baseline artifacts at 214 and 247 bases observed in the JOE dye channel [1-2].

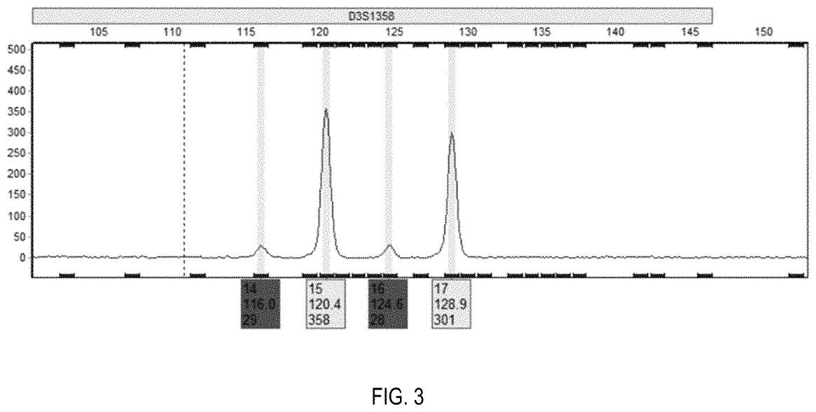

Stutter filters will be applied using the methodology in FIG. 3 and Table 1.

There is seen in FIG. 3 an example of a stutter calculation used in the iLSST-NR system. This example demonstrates the method used to calculate the stutter-corrected peak heights when using either the internally developed stutter models or the manufacturer's recommended stutter filters. Note, in this example a static stutter rate of 10% for reverse and 1% for forward stutter will be used for demonstrative purposes only.

TABLE 1

| | Starting | Final | | |
|---|---|---|---|---|
| | peak height | peak height | | |
| Allele | (RFU) | (RFU) | Calculation | Notes |
| 14 | 29 | −6.8 | 29 − | peak |
| | | | (358*0.1) | zeroed |
| 15 | 358 | 354.91 | 358 − | |
| | | | (28*0.1) − | |
| | | | (29*0.01) | |
| 16 | 28 | −5.68 | 28 − | peak |
| | | | (301*0.1) − | zeroed |
| | | | (358*0.01) | |
| 17 | 301 | 300.72 | 301 − | |
| | | | (28*0.01) | |

Trimming Algorithms

Trimming algorithms were employed to decrease noise due to non-allelic, non-stutter related peaks. The minimum:maximum ratio and maximum locus-global minimum trimming algorithm are used in tandem to eliminate low level noise not due to additional contributors. These algorithms are rule-based and can be tuned by the user if needed.

Maximum Locus-Global Minimum

The locus or loci that has the largest number of potential allelic peaks can also be considered to have the highest information content in the sample; we have termed these loci “maximum loci”. The algorithm identifies the smallest peak at the “maximum locus” and will trim any peak at other loci in the samples that are not within 5.0% of the peak height of the previously identified maximum locus peak. If several maximum loci are present, the mean peak height of the lowest peak at these loci is used. The default value of 0.05 has shown to be effective at trimming noise from samples amplified using the PowerPlex Fusion® DNA amplification kit (Promega Corporation). Other commercial or non-commercial multiplexes may exhibit peak height imbalance or noise that differs from the PowerPlex Fusion amplification kit. We recommend that the maximum locus-global minimum trimming value be empirically determined to avoid trimming of allelic peaks associated with low minor contributors.

Minimum:Maximum Ratio

This locus-specific trimming algorithm will remove aberrant alleles that are outside of a user specified proportion of the highest peak height at a locus. This minimum:maximum proportion threshold was empirically determined and was set at 0.019 for this study, meaning any signal above the LSST but not within 1.9% of the height of the highest peak at a locus will be trimmed. This level appropriately balanced the removal of noise and the retention of low-level allelic activity.

Machine Learning

All samples were randomly partitioned such that approximately 75% (960 samples) were placed within a training set and the remaining approximately 25% (341 samples) within a testing set. A support vector machine (SVM) was used to learn a predictive model capable of classifying a locus as either containing or not containing one or more artifacts. Feature extraction and construction of the feature vector were performed using privately-developed software written in the Python programming language using the scikit-learn library. All SVM hyperparameter tuning was performed using a grid search and validated using 5-fold cross-validation on the training set. Raw SVM outputs were calibrated using isotonic regression to estimate probabilities.

NOC-Violating Trimming

A precursor machine learning algorithm was used to learn a basic predictive model capable of estimating the probabilities that the number of contributors in a given sample is 1, 2, 3, and 4 or more, respectively. After probabilities are determined for each possible allowed number of contributors for a given sample, the highest probability is used to set a temporary required number of contributors. For each locus in the sample, if the number of peaks is greater than twice the temporary required number of contributors, superfluous peaks are evaluated from smallest to largest. For each peak, if the peak height is less than or equal to three times the dynamic threshold value for the locus, the peak is trimmed.

Artifact Trimming

After machine learning as described in section 2.5 has been completed, the resulting model estimates the probability that a given locus contains one or more artifacts. Each peak at the locus (excluding the largest peak) is evaluated, from smallest to largest. If a peak's height is smaller than a pre-defined “high-template threshold”, and is less than two times the dynamic threshold value for the locus, and if the model-derived probability that an artifact is present in the locus is greater than or equal to 0.99, that peak is trimmed.

Feature Selection and Feature Engineering

The use of high-value features in machine learning allows one to maximize the predictive value of the algorithm. The feature's classification “importance” can be estimated using Kullback-Leibler divergence (Equation 3), which is a measure of the reduction in entropy of the class variable (in this case, the true number of contributors) after the value for the feature is observed.

$${D}_{KL} ( P \vert \vert Q ) = {\sum }_{i} P ( i ) log \frac{P ( i )}{Q ( i )} . \qquad ( 3 )$$

All candidate features were ranked by divergence, and any candidate feature with a divergence below 0.01 was removed prior to machine learning. Calculations of Kullback-Leibler divergence were performed using the Weka Knowledge Analysis Environment, version 3.8. Some of the features were “native”, and taken directly from the electropherogram, while others were “derived” and resulted from various manipulations of raw data. For example, features involving the probability that the DNA sample contained a given number of contributors were obtained using a precursor machine learning algorithm.

Analyses

The performance of the Modules 1 through 4 was evaluated through a comparison to a dynamic threshold without trimming algorithms, a 50 RFU static threshold with and without trimming algorithms, a 100 RFU static threshold with and without trimming algorithms, and a 150 RFU static threshold with and without trimming algorithms. The methods that use static thresholds without the trimming algorithms are the ones most commonly used across the forensic DNA community. Performance was evaluated using (1) system induced dropout—the alleles that are above 10 RFU but are trimmed by the threshold or trimming algorithms and (2) additional unexpected alleles present. Percent accuracy (Equation 4) was used to demonstrate the effectiveness of the system's ability to decrease artificial dropout (dropout due to the application of the threshold and trimming algorithms).

$$percent accuracy = \frac{number of correct allele calls}{total number of expected allele calls} \times 100 \qquad ( 4 )$$

The allele detection and noise reducing methods were compared using the stutter models as well as stock stutter values provided by the Promega Corporation [38, 45]. Although the sample set was comprised of 1301 samples, it was necessary to evaluate the methods using the 341 samples in the testing subset. This avoids any bias that may be present in the training set for those systems that utilize machine learning-derived models. The overall system performance was compared to the performance of the 50, 100 and 150 RFU static thresholds with stock stutter filters using precision, recall, F-score and informedness:

Precision (Equation 5) is a measure of confidence, also known as the positive predictive value. In the context of allele or artifact detection, precision represents the proportion of correctly identified artifacts (or alleles) to the total number of artifacts (or alleles) predicted.

$$precision = \frac{number of correctly predicted positive events}{number of predicted positive events} \qquad ( 5 )$$

Recall (Equation 6), also known as the true positive rate or sensitivity, represents the predicted rate of positive identification for the specific class. In the context of this study, recall represents the proportion of correctly predicted alleles (or artifacts) to the total number of alleles (or artifacts) expected.

$$recall = \frac{number of correctly predicted positive events}{number of positive events} \qquad ( 6 )$$

The F1 score (Equation 7) is the harmonic mean of precision and recall. Generally, predictive systems with F1 values approaching one will display higher accuracy. The global F1 score (Equation 8) assesses the performance of a method across classes (detection of true alleles and detection of artifacts) without attempting to weight or normalize classes by class frequency in the training data.

$$\begin{matrix}F 1 = 2 \times ( \frac{precision \times recall}{precision + recall} ) & ( 7 ) \\ global F 1 = \frac{{\sum }_{i = 1}^{n} F {1}_{i}}{n} & ( 8 )\end{matrix}$$

Informedness (Equation 9) represents the level of confidence that the system has in predicting the class. In this case, informedness represents the relative level of confidence the system has in accurately trimming an allele.

informedness=recall+(recall⁻¹)−1 (9)

Learning curves, which plot prediction accuracy as training size is varied, were generated for all machine learning-derived models using 10-fold cross-validation and were plotted with a band of +/−one standard deviation from each point. In addition, a receiver operating characteristic (ROC) curve, a graphical representation of the learner's performance as its discrimination threshold is varied, was generated to assess the resulting model's diagnostic ability.

Results

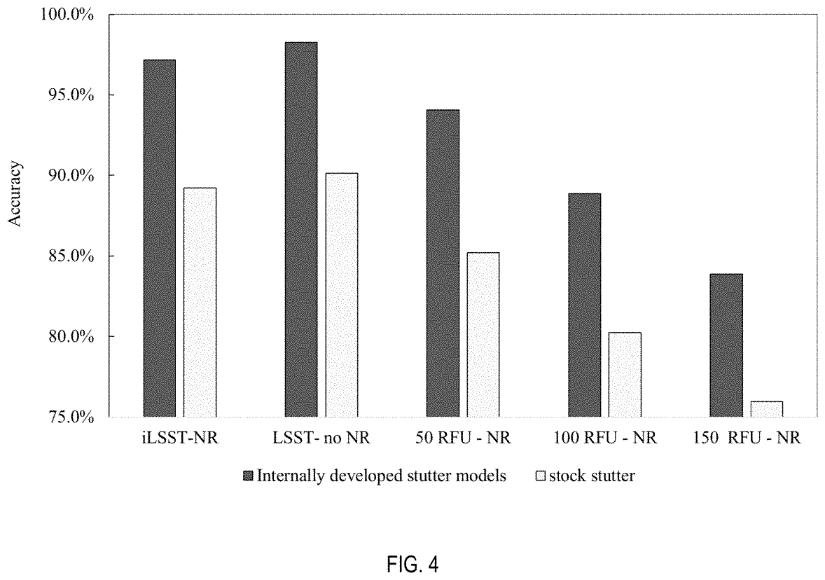

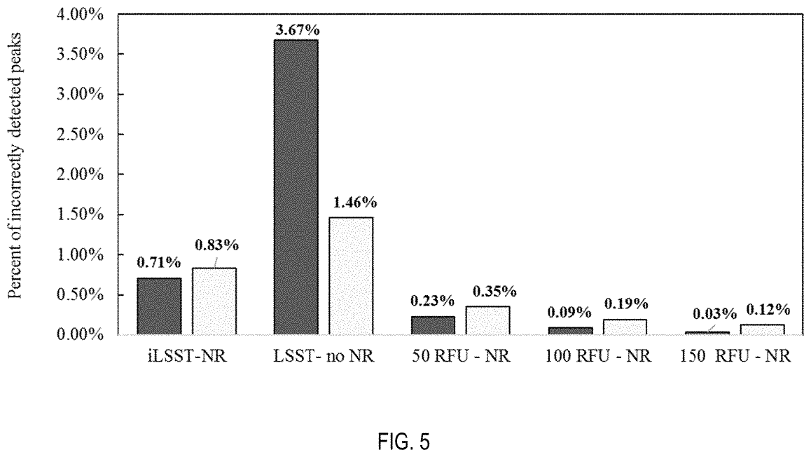

The iLSST-NR method using modeled stutter filters outperforms all other methods in the ability to maintain a high level of information content—balancing false positives (noise that has been “called”) and false negatives (threshold/trimming driven allelic dropout). Overall, the system had a 97.2% success rate in detecting alleles (583 instances of dropout across 20,662 expected alleles) (Table 2 and FIG. 4). In addition, only 0.79% of the detected peaks were non-allelic, 142 out of 20,079 detected peaks across 95 samples (Table 2 and FIG. 5). The trimming algorithms have a clear positive impact on the detection of unexpected, non-allelic peaks, with a minimum 3.8-fold reduction in the calling of incorrect or aberrant peaks. The LSST (module 1) led to the incorrect detection of 746 but was decreased by 604 or 81% when processed through the downstream trimming methods (modules 3 and 4). As expected, the number of incorrect remaining alleles decreased and the number of dropout alleles increased when increasing static thresholds of 50, 100 and 150 RFU were applied. Threshold induced allele dropout was lowest (1.8%) when applying the locus-sample-specific threshold without trimming; however, it had more than a 5-fold increase in incorrect alleles detected compared the iLSST-NR system.

The performance of the various thresholding and noise reducing systems using internally developed stutter models and stock stutter. Note that the 50, 100 and 150 RFU thresholds with stock stutter filters are present to allow a comparison to currently used thresholding methods.

TABLE 2

| | | | | | Percent- |
|---|---|---|---|---|---|
| Threshold/ | | | | Incorrect | age of |
| trimming | Stutter | Dropout | Accu- | remaining | additional |
| method | filter | alleles | racy | alleles | alleles |
| iLSST-NR | modeled | 583 | 97.2% | 142 | 0.79% |
| LSST | modeled | 362 | 98.2% | 746 | 3.67% |
| 50 RFU - NR | modeled | 1225 | 94.1% | 44 | 0.23% |
| 50 RFU | modeled | 1225 | 94.5 | 181 | 0.93 |
| 50 RFU | stock | 3004 | 85.5% | 116 | 0.66% |
| 100 RFU - NR | modeled | 2301 | 88.9% | 16 | 0.09% |
| 100 RFU | modeled | 2290 | 88.9 | 84 | 0.29 |
| 100 RFU | stock | 4059 | 80.4% | 51 | 0.31% |
| 150 RFU - NR | modeled | 3330 | 83.9% | 6 | 0.03% |
| 150 RFU | modeled | 3359 | 83.7 | 23 | 0.13 |
| 150 RFU | stock | 4957 | 76.0% | 31 | 0.20% |

Of the 142 non-allelic peaks incorrectly called, 127 (88.7%) were located in forward or reverse stutter position (Table 2). The remaining 16 peaks span 11 loci, in mixtures of 1- to 4-contributors and were run on both the 3500 and 3100 series of instruments with varied injection times and voltages (Table 3). It is noteworthy, however, that 6/16 were amplified using 1.0 ng or greater (injection time=5 s and injection voltage=3 kV), above the manufacturer's 0.5 ng recommendation and where there can be higher incidence of elevated baseline [38, 45]. Samples 3 and 5 contain clear baseline artifacts that would likely be either re-injected or rerun. We routinely observed elevated baseline artifacts in the 9 and 10 allele positions of D3S1358; only one instance was called by the iLSST-NR system, a 10 allele in Sample 9. The remaining calls may be the result of poor migration (Sample 8), possible allelic drop-in (samples 4 and 13), elevated baseline noise due to proximity to the primer peak (samples 15 and 16) or are commonly observed artifacts (sample 9).

TABLE 3
Baseline or artifacts (not in stutter position) that were erroneously identified as
peaks by iLSST-NR.

| | | | Additional | DNA | | | |
|---|---|---|---|---|---|---|---|
| | | | incorrect | template | | Injection | |
| | | | called | amplified | Injection | voltage | Instrument |
| Sample | Locus | Genotype | allele | (ng) | time (s) | (kV) | type |
| Sample 1 | CSF1PO | (11, 12)(10, 13) | 7 | 1 | 5 | 3 | 3130 |
| Sample 2 | CSF1PO | (11, 12)(11, 12) | 7 | 1 | 5 | 3 | 3130 |
| Sample 3 | D10S1248 | (14, 15)(14, 15) | 12 | 0.1 | 24 | 1.2 | 3500 |
| Sample 4 | D12S391 | (15,21) | 24 | 4 | 5 | 3 | 3500 |
| Sample 5 | D12S391 | (15,21) | 24 | 4 | 5 | 3 | 3100 |
| Sample 6 | D16S539 | (11, 12)(11, | 7 | 0.45 | 10 | 1.2 | 3500 |
| | | 13)(11, 12) | | | | | |
| Sample 7 | D19S433 | (15.2, 16.2) | 11 | 4 | 5 | 3 | 3100 |
| Sample 8 | D2S441 | (10, 11)(10, | 11.3 | 0.5 | 18 | 1.2 | 3500 |
| | | 10)(15, 15) | | | | | |
| | | (11, 13.3) | | | | | |
| Sample 9 | D3S1358 | (15, 17) | 10 | 0.125 | 5 | 3 | 3100 |
| Sample 10 | D3S1358 | (17, 17)(18, | 13 | 0.5 | 5 | 3 | 3130 |
| | | 18)(16, 16) | | | | | |
| Sample 11 | D3S1358 | (17, 17)(18, | 13 | 1 | 5 | 3 | 3130 |
| | | 18)(16, 16) | | | | | |
| Sample 12 | D7S820 | (8, 10)(9.1, 11) | 14 | 0.25 | 5 | 3 | 3130 |
| Sample 13 | DYS391 | N/A-female | 10 | 0.25 | 5 | 3 | 3100 |
| Sample 14 | FGA | (18, 20)(21, | 31.2 | 0.45 | 10 | 1.2 | 3500 |
| | | 26)(20, 22) | | | | | |
| Sample 15 | TH01 | (9, 9.3)(7, 10) | 3 | 0.45 | 10 | 1.2 | 3500 |
| | | (6, 9) | | | | | |
| Sample 16 | TH01 | (6, 9)(9, 9) | 4 | 0.1 | 5 | 3 | 3100 |

The iLSST-NR system outperforms the static thresholds with a global F1 score of 0.976, 7.5% higher than any other method (Table 4). The system also yields the highest class-specific F1 scores, 0.982 and 0.97 for the allele and artifact classes, respectively. The precision and recall values remain consistently above 0.95 across classes, in contrast to the static thresholds, which have increasingly disparate values as the RFU level is increased. This inverse relationship is driven by decreased precision when classifying peaks as artifacts and similar decreases in recall in the allele class, as an approximately 3% decrease is observed with every 50 RFU increase in the threshold.

TABLE 4
Summary statistics for thresholding and noise reducing systems.

| Trimming | | | | | Global | |
|---|---|---|---|---|---|---|
| method | Class | Precision | Recall | F1 | F1 | Informedness |
| iLSST-NR | Allele | 0.993 | 0.972 | 0.982 | 0.976 | 0.924 |
| | Artifact | 0.952 | 0.988 | 0.970 | | 0.981 |
| 50 RFU | Allele | 0.993 | 0.855 | 0.919 | 0.901 | 0.649 |
| | Artifact | 0.794 | 0.990 | 0.882 | | 0.984 |
| 100 RFU | Allele | 0.997 | 0.804 | 0.890 | 0.87 | 0.545 |
| | Artifact | 0.742 | 0.996 | 0.850 | | 0.993 |
| 150 RFU | Allele | 0.998 | 0.760 | 0.863 | 0.844 | 0.462 |
| | Artifact | 0.702 | 0.997 | 0.824 | | 0.995 |

Stutter

The performance of the thresholding systems were compared with both the internally developed forward and reverse stutter models and the stock stutter rates (Table 5). The modeled stutter filters dramatically improved the performance of allele detection and noise reduction. The systems using modeled stutter filters had an average increase of 1720 expected alleles called (8.35%), dramatically reducing the incidence of threshold-induced allelic dropout. The detection of incorrect alleles was affected less by the choice in stutter filters. Five systems had fewer incorrect alleles when using modeled stutter and the remaining three favored the use of the stock stutter filters. The LSST and the 50 RFU systems were significantly impacted by the use of modeled stutter filters with an additional 471 and 65 incorrect alleles, respectively. The remaining systems had an average 0.09% change in the number of incorrect alleles detected. The iLSST-NR system with modeled stutter filters detected 97.2% of the true alleles, and only 142 (0.71%) additional peaks were erroneously classified as alleles. The majority of incorrectly detected peaks, 126/142 (88.7%), were in stutter position ((0.6% (126/20,0072 across the complete data set). Those in forward stutter position accounted for 38/142 (26.8%), 56/142 (39.4%) were in reverse stutter position, and 32/142 (22.5%) were in a combined forward and reverse stutter position. Only 16/142 (11.2%) incorrectly identified noise peaks were not in a stutter position.

TABLE 5
The performance of the various thresholding and noise reducing
systems using modeled or stock stutter filters.

| | | | | | Percent- |
|---|---|---|---|---|---|
| Threshold/ | | | | Incorrect | age of |
| trimming | Stutter | Dropout | Accu- | remaining | additional |
| method | filter | alleles | racy | alleles | alleles |
| iLSST-NR | modeled | 583 | 97.2 | 142 | 0.71 |
| iLSST-NR | stock | 2248 | 89.2 | 154 | 0.83 |
| LSST | modeled | 362 | 98.2 | 746 | 3.67 |
| LSST | stock | 2060 | 90.1 | 275 | 1.46 |
| 50 RFU - NR | modeled | 1225 | 94.1 | 44 | 0.23 |
| 50 RFU - NR | stock | 3059 | 85.2 | 61 | 0.35 |
| 50 RFU | modeled | 1225 | 94.5 | 181 | 0.93 |
| 50 RFU | stock | 3004 | 85.5 | 116 | 0.66 |
| 100 RFU - NR | modeled | 2301 | 88.9 | 16 | 0.09 |
| 100 RFU - NR | stock | 4085 | 80.2 | 32 | 0.19 |
| 100 RFU | modeled | 2290 | 88.9 | 84 | 0.29 |
| 100 RFU | stock | 4059 | 80.4 | 51 | 0.31 |
| 150 RFU - NR | modeled | 3330 | 83.9 | 6 | 0.03 |
| 150 RFU - NR | stock | 4966 | 76.0 | 19 | 0.12 |
| 150 RFU | modeled | 3359 | 83.7 | 23 | 0.13 |
| 150 RFU | stock | 4957 | 76.0 | 31 | 0.20 |

Trimming Algorithms

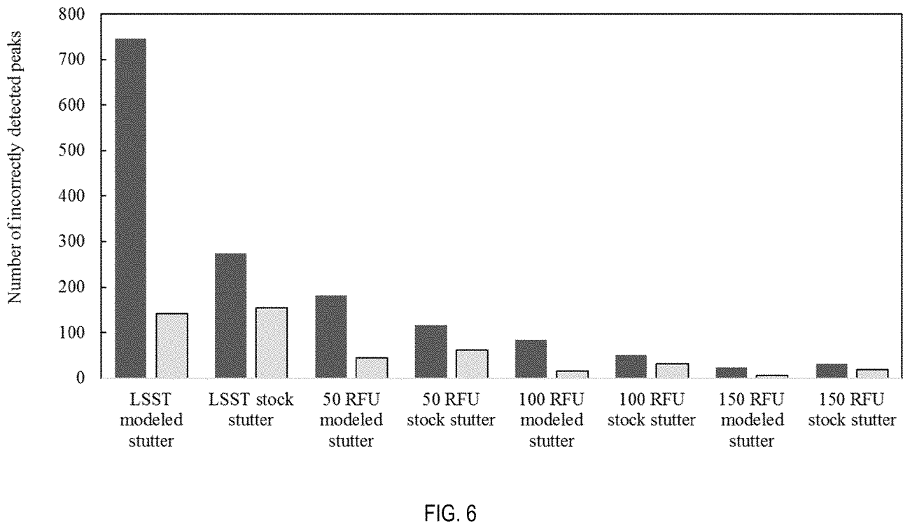

The use of the trimming algorithms (from modules 3 and 4) led to a significant reduction in the number of incorrectly called alleles, with an average 3-fold decrease when trimming is used (Table 5 and FIG. 6). Trimming had the highest impact on the LSST with modeled stutter (iLSST-NR), with a 5.25-fold decrease in the number of incorrect calls. This further supports the use of trimming with the dynamic threshold.

The minimum:maximum ratio trimming correctly removed 543 peaks that were non-allelic, with 194 peaks incorrectly trimmed. These incorrectly trimmed peaks account for 33.3% (194/583) of the total dropout observed. The maximum locus-global minimum trimming correctly removed one non-allelic peak and, similarly, incorrectly trimmed only one true peak.

Instrument Comparison

The overall data set is comprised of data from five laboratories and both the 3500 and 3100 series instruments. This comparison partitions the data set into instrument specific subsets due to the 3- to 4-fold increase in RFU scale between the instruments. The iLSST-NR method maintains the highest level of information content without the introduction of significant amounts of aberrant non-allelic peaks (Table 6 and Appendix A-Supplementary Information Tables 3A and 4A). The highest accuracy for both the 3100 and 3500 series was obtained when using the modeled stutter filters with the dynamic threshold without trimming, 96.3% and 99.1% respectively; however, the counterpart with trimming (iLSST-NR) demonstrated nearly equivalent accuracies of 94.6% and 98.4%, respectively. The iLSST-NR method has 236 fewer incorrectly called alleles than the dynamic threshold with no trimming.

TABLE 6
The performance of the thresholding and noise reducing systems based on
instrument, 3100 series and 3500 series. Note, the results
reflect the use of internally developed stutter models.

| | | | | | Incorrect | Percentage |
|---|---|---|---|---|---|---|
| Instrument | | Trimming | Dropout | | remaining | additional |
| series | stutter | method | alleles | Accuracy | alleles | alleles |
| 3500 | modeled | iLSST-NR | 362 | 94.6% | 43 | 0.67% |
| | | LSST no NR | 250 | 96.3% | 279 | 4.30% |
| | stock | 50 RFU | 944 | 86.0% | 46 | 0.79% |
| | | 100 RFU | 1091 | 83.8% | 45 | 0.80% |
| | | 150 RFU | 1224 | 81.8% | 28 | 0.51% |
| 31xx | modeled | iLSST-NR | 220 | 98.4% | 99 | 0.72% |
| | | LSST no NR | 130 | 99.1% | 551 | 3.99% |
| | stock | 50 RFU | 2103 | 84.9% | 30 | 0.25% |
| | | 100 RFU | 2968 | 78.7% | 6 | 0.05% |
| | | 150 RFU | 3732 | 73.2% | 3 | 0.03% |

Supplemental Machine Learning Evaluation

All candidate features for the machine learning's feature vector were evaluated using Kullback-Leibler divergence. A subset of the features used in this analysis are found in Table 7. The highest value recorded was 0.091; no features with divergences below 0.01 were retained for subsequent machine learning.

TABLE 7
Kullback-Leibler divergence for some of the candidate features
used. Note, Pr(NOC) refers to the probability that a particular
sample or locus is 1-, 2-, 3- or 4-contributors; maximum
peaks refers to the number of peaks at the locus with the
maximum number of peaks across the sample.

| Feature | D_{KL} |
|---|---|
| Ratio of minimum peak height | 0.091 |
| to maximum peak height | |
| Minimum intra-locus peak | 0.045 |
| height | |
| Pr(NOC = 4) | 0.043 |
| Pr(NOC = 3) | 0.038 |
| Pr(NOC = 2) | 0.037 |
| Number of peaks at a locus | 0.037 |
| Maximum peaks at a locus | 0.023 |
| across the entire sample | |

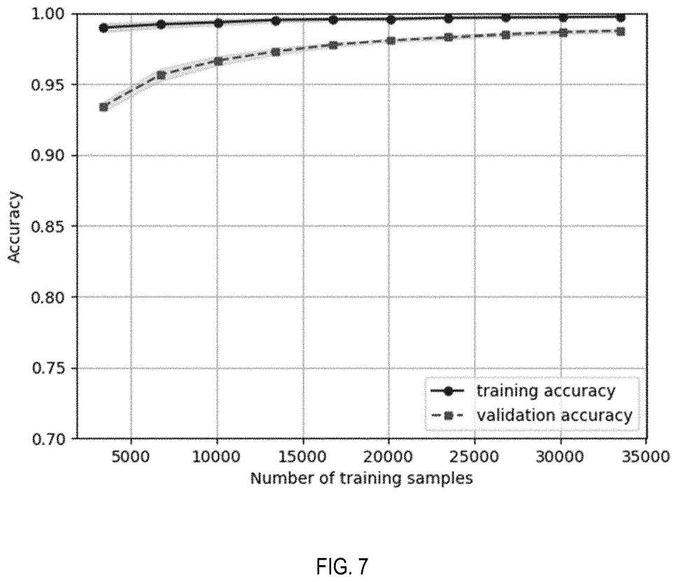

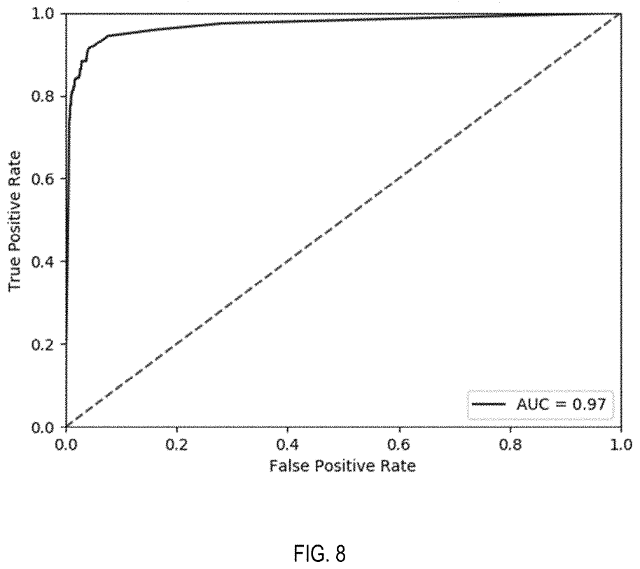

Plots were also generated to evaluate algorithm generalization, learning, and performance. The algorithm's learning curve exhibited a final training accuracy of 0.998 and a final validation accuracy of 0.982, with non-overlapping standard error bands for both curves. Accuracy failed to increase by 1% per 1000 samples when the sample size had reached 30000 samples (FIG. 7). The ROC curve exhibited an area under the curve of 0.97 (FIG. 8).

Machine learning-assisted noise reduction resulted in the correct removal of 60 non-allelic peaks and only 8 true alleles, an accuracy of 88.2%. The eight incorrectly removed alleles were alleles attributed to the minor contributor of 3-contributor samples (Table 8). These samples had low-level minor components that accounted for approximately 0.071 ng or less of the total template DNA amplified.

TABLE 8
Samples where one true allele was incorrectly removed due to
the machine-learning based trimming. This accounted for only
8 instances of incorrect trimming across 1301 total samples.

| | | | | Estimated tem- |
|---|---|---|---|---|
| | | | Template | plate associated |
| | | Number of | DNA | with minor |
| | Mixture | contrib- | amplified | contributor |
| Sample | ratio | utors | (ng) | (ng) |
| Sample 17 | 10:4:1 | 3 | 0.45 | 0.03 |
| Sample 18 | 5:5:1 | 3 | 0.1 | 0.009 |
| Sample 19 | 5:1:1 | 3 | 0.5 | 0.071 |
| Sample 20 | 5:1:1 | 3 | 0.5 | 0.071 |
| Sample 21 | 20:1:1 | 3 | 0.5 | 0.023 |
| Sample 22 | 20:1:1 | 3 | 0.5 | 0.023 |
| Sample 23 | 20:1:1 | 3 | 0.5 | 0.023 |
| Sample 24 | 20:1:1 | 3 | 0.5 | 0.023 |

Mixtures

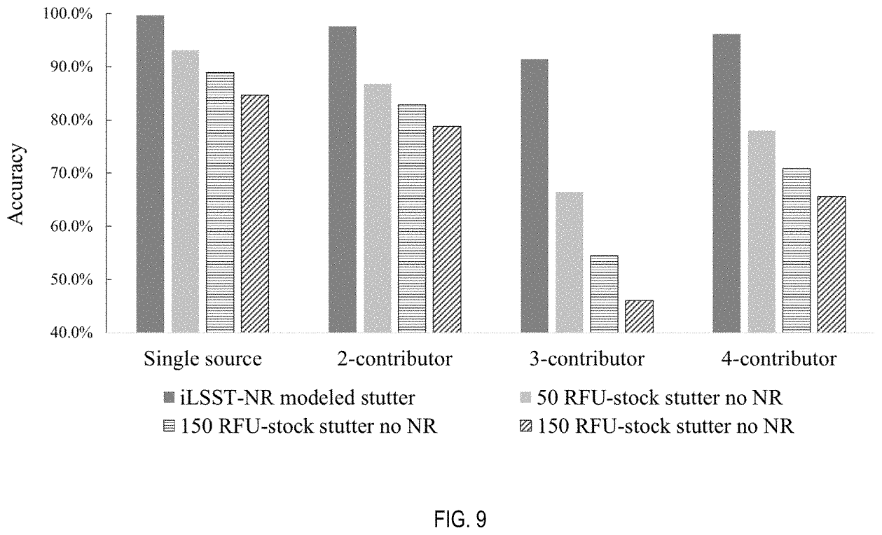

The number of contributors in a DNA sample may impact the baseline noise levels. Additional allelic activity may lead to an increase in baseline noise as well as introduce additional stutter artifacts. The performance of the iLSST-NR with modeled stutter system was compared to three standard thresholding methods (50, 100 and 150 RFU) using stock stutter rates (FIG. 9 and Appendix A—Supplementary Information—Table 6A). We observed a drop of 2.1% (iLSST-NR) to 6.3% (50 RFU-noNR stock stutter) accuracy from a single source to a 2-contributor sample. The decrease in accuracy from two contributors to three contributors ranges from 6.1% (iLSST-NR) to 19.0% (150 RFU). An increase in accuracy was observed between 3- and 4-contributor samples. The overall drop in accuracy from single source to 4-contributor samples is 3.6%, 15.1%, 18.1%, and 19% for iLSST-NR, 50 RFU-stock stutter, 100 RFU-stock stutter and 150 RFU-stock stutter, respectively. The average change in accuracy as contributor number is increased is 1.2%±0.05, 5.03%±0.16, 6.03%±0.22 and 6.33%±0.26 for iLSST-NR, 50 RFU-stock stutter, 100 RFU-stock stutter and 150 RFU-stock stutter, respectively.

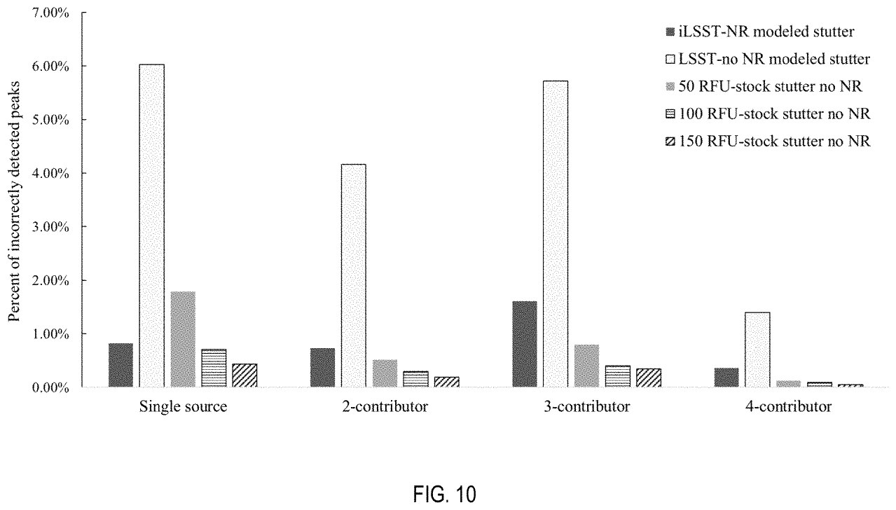

There was an inverse relationship between the threshold level and the number of incorrect allele calls. However, unlike the accuracy rate for correctly identifying true alleles, we did not observe a clear trend in the percentage of incorrect alleles called across the number of contributors (FIG. 10A). It is noteworthy that the 4-contributor samples yielded the lowest overall percent of additional alleles called across all thresholding systems. The iLSST-NR system experienced a higher percentage of incorrect additional alleles being called (0.79%), although overall performance of the iLSST-NR system as measured by global F1 was 7.5% higher than the highest-performing static threshold.

When the present invention was applied to 3480 sample, the system predicted dropout correctly 87.60% of the time (3047 correct and 433 incorrect).

The present invention described or otherwise envisioned herein is proposed as a valuable tool in the analyst assessment of the occurrence of allelic dropout. As described, the invention utilizes a machine learning approach to identify and assign probability estimates to the presence of allelic dropout in a genetic sample. This method further utilizes a more expansive set of data categories than current methods. The proposed method includes features including, but not limited to, the number of alleles observed across the sample, the number of alleles at a particular locus within the marker set, estimated number of contributors to the sample using the maximum allele count method, DNA template, the average contribution of the alleles to a sample and/or locus, the traditional dropout probability (using known DNA samples, a model generated by plotting the presence of allele dropout and the average allelic contribution or template DNA concentration), and inter- and intra-locus maximum and minimum allelic contributions. A machine learning algorithm then is trained using known samples and the previously mentioned data categories (features); the resulting model will then permit an assessment of the probability of allelic dropout in a specific DNA locus in an unknown sample.

According to an embodiment, the invention is a system configured to assess the occurrence of allele dropout in a sample or DNA locus within a sample. The system includes: a sample preparation module configured to generate initial data about the DNA within the sample; a processor comprising a allele dropout determination module, wherein the presence of allele dropout determination module comprises a machine-learning algorithm configured to: (i) receive the generated initial data; (ii) analyze the generated initial data to determine the presence of allele dropout within the sample; and an output device configured to receive the determined occurrence of allele dropout from the processor, and further configured output information about the received determined occurrence of allele dropout.

According to an embodiment, the machine-learning algorithm comprises a support vector machine algorithm.

According to an embodiment, the output device comprises a monitor.

According to an embodiment, the sample preparation module comprises amplification of DNA within the sample. According to an embodiment, the sample preparation module comprises amplification of one or more DNA markers within the sample.

According to an aspect is a system configured to characterize the occurrence of allele dropout in DNA within a sample, the system comprising a processor configured to receive data about the DNA within the sample, and further configured to analyze, using a machine-learning algorithm, the received data to determine the presence of allele dropout to the DNA within the sample

According to an embodiment, the system further includes a sample preparation module configured to generate the data about the DNA within the sample. According to an embodiment, the sample preparation module comprises amplification of DNA within the sample. According to an embodiment, the sample preparation module comprises amplification of one or more DNA markers within the sample.

According to an embodiment, the system further includes an output device in communication with the processor, the output device configured output information about the received determined presence of allele dropout. According to an embodiment, the output device comprises a monitor.

According to an embodiment, the machine-learning algorithm comprises a support vector machine algorithm.

According to an aspect is a method for characterizing the occurrence of allele dropout with in a DNA sample or a DNA mixture within a sample. The method comprises the steps of: (i) generating, using a sample preparation module, initial data about the DNA within the sample; (ii) receiving, by a processor comprising an allelic dropout determination module executing a machine-learning algorithm, the generated initial data; (iii) analyzing, by the allelic dropout determination module executing a machine-learning algorithm, the generated initial data to predict the occurrence of allele dropout within the DNA sample; and (iv) providing, by an output device configured to receive the predicted occurrence of allele dropout from the processor, information about the received predicted occurrence of allele dropout.

According to one embodiment, the system can comprise a single unit with one or more modules, or may comprise multiple modules in more than one location that may be connected via a wired and/or wireless network connection. Alternatively, information may be moved by hand from one module to another. The system may be implemented by hardware and/or software, including but not limited to a processor, computer system, database, computer program, and others. The hardware and/or software can be implemented in different systems or can be implemented in a single system.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

A “module” or “component” as may be used herein, can include, among other things, the identification of specific functionality represented by specific computer software code of a software program. A software program may contain code representing one or more modules, and the code representing a particular module can be represented by consecutive or non-consecutive lines of code.

As will be appreciated by one skilled in the art, aspects of the present invention may be embodied/implemented as a computer system, method or computer program product. The computer program product can have a computer processor or neural network, for example, that carries out the instructions of a computer program. Accordingly, aspects of the present invention may take the form of an entirely hardware embodiment, an entirely software embodiment, and entirely firmware embodiment, or an embodiment combining software/firmware and hardware aspects that may all generally be referred to herein as a “circuit,” “module,” “system,” or an “engine.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction performance system, apparatus, or device.

The program code may perform entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The flowcharts/block diagrams in the Figures illustrate the architecture, functionality, and operation of possible implementations of systems, methods, and computer program products according to various embodiments of the present invention. In this regard, each block in the flowcharts/block diagrams may represent a module, segment, or portion of code, which comprises instructions for implementing the specified logical function(s). It should also be noted that, in some alternative implementations, the functions noted in the block may occur out of the order noted in the figures. For example, two blocks shown in succession may, in fact, be performed substantially concurrently, or the blocks may sometimes be performed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

What is claimed is:

1. A system configured to characterize allele dropout, comprising:
a database containing sequence data representing DNA present in a sample;
a processor coupled to the database and configured to receive the sequence data representing DNA in the sample from the database, wherein the processor is programmed to predict the occurrence of any allelic dropout at a given locus by applying an allelic dropout model to assess predetermined categorical and quantitative aspects of the sequence data based on at least one feature in the sequence data; and
an output device configured to receive the predicted occurrence of allele dropout from the processor and provide the predicted occurrence to a user.

2. The system of claim 1, wherein the machine-learning algorithm is a support vector machine algorithm.

3. The system of claim 1, wherein the output device is a monitor.

4. The system of claim 1, further comprising a sample preparer is configured to generate the sequence data about DNA within the sample.

5. The system of claim 4, wherein the sample preparer is configured to amplify DNA within the sample.

6. The system of claim 5, wherein the sample preparation module is configured to amplify at least one DNA marker within the sample.

7. The system of claim 1, wherein the database contains sequence data representing DNA present in a known sample with predetermined allelic dropout probabilities and the processor is further programmed to receive sequence data representing DNA present in the known sample and assess the sequence data representing DNA present in the known sample to develop a model for predicting allelic dropout in an unknown sample.

8. The system of claim 8, wherein the processor is programmed to use machine learning to evaluate a plurality of features in the sequence data representing DNA present in the known sample and to develop an allelic dropout model for determining the probability of allelic dropout.

9. A method of characterizing any occurrence of allele dropout in a sample, comprising the steps of:
using a sample preparer to generate sequence data for any DNA within the sample;
receiving the sequence data with a processor configured to receive the sequence data;
using the processor to predict the occurrence of any allelic dropout at a given locus in the sequence data by applying a predetermined allelic dropout model to assess at least one feature of the sequence data representing categorical and quantitative aspects of the sequence data;
using an output device to receive the predicted occurrence of allele dropout from the processor and provide information about the received predicted occurrence of allele dropout to a user.

10. The method of claim 9, further comprising the step of creating the predetermined allelic dropout model using a machine-learning algorithm.

11. The method of claim 10, wherein the machine-learning algorithm is a support vector machine algorithm.

12. The method of claim 11, wherein the output device is a monitor.

13. The method of claim 9, further comprising the step of using a sample preparer to generate the sequence data for any DNA within the sample.

14. The method of claim 13, wherein the step of using a sample preparer includes amplification of any DNA within the sample.

15. The method of claim 14, wherein the step of using a sample preparer includes amplification of one or more DNA markers within the sample.
